(19) United States
(12) Patent Application Publication
(10) Pub. No.: US 2008/0138875 A1
(43) Pub. Date: Jun. 12, 2008

(54) Method to generate fungal biomass from a culture of differentiated mycelium

(76) Inventors: Lucia Atehortua, Medellin (CO); Paola Andrea Zapata, Medellin (CO); Diego Fernando Rojas, Envigado (CO); David Alexander Ranirez, Medellin (CO); Carlos Fernandez, Antioquia (CO)

Correspondence Address: JOHN J. MARTINEZ MD. JD., 10 PALMER AVENUE, CROTON ON HUDSON, NY, 10520, US

(21) Appl. No.: 11/635,986

(22) Filed: Dec. 8, 2006

(51) Int. Cl. C12P 1/02; C12M 1/00; C12N 1/14

(52) U.S. Cl. 435/171; 435/292.1; 435/256.8

(57) ABSTRACT

The present invention provides method to produce fungi biomass from mycelia comprising inoculating differentiated cells or mycelia into a culture medium which contains at least NaNO₃, KH₂PO₄, MgSO₄, KCl, and a source of carbon. This method also includes incubation of said culture under a specific wave length in a specially designed tridimensional closed system or incubator in which all the internal surfaces have all the characteristics of a mirror.

### BACKGROUND OF THE INVENTION

1. Field of the Invention

In the field of the present invention, the medicinal fungi Ganoderma lucidum has been commercially cultured in solid state using substrates such as agricultural waste, cereals and wood. Cultures in solid state take 2 to 8 months and yields are low.

In recent years, submerged fermentation cultures in Erlenmeyer's flasks escalated to bioreactors have surfaced as a more efficient alternative for the production of mycelia biomass. However, the cost still remains high and the demand for these medicinal fungi is increasing.

2. Description of Prior Art

In the prior art, the most efficient production of mycelia biomass for Ganoderma lucidum has been reported by Tang & Zong (see Tang, Y. J., Zhong, J. J., Enzyme and Microbial Technology, 31:20-28 (2002); Tang, Y. J., Zhong, J. J., Biotechnology Letters, 24:1023-1026 (2002)). These authors reported submerged fermentation cultures with a yield of 22.1 g/L, and a culture time of 14-17 days, using submerged cultures or liquid fermentation.

The present invention overcomes the limitations of solid and liquid fermentation, and submerged cultures. In addition, the present invention provides a method for the production of biomass from fungi mycelia, wherein said method reduces the cost more than 60 times. Moreover, the method of the present invention increases the yield per day of biomass. Furthermore, said biomass has a better or comparable exo-polysaccharides, intra-polysaccharides, and ganoderic acid composition.

### SUMMARY OF THE INVENTION

The present invention provides method to produce fungi biomass from mycelia comprising inoculating differentiated cells or mycelia into a culture medium which contains at least NaNO₃, KH₂PO₄, MgSO₄, KCl, and a source of carbon; and, incubating said culture medium with differentiated cells or mycelia in a container with transparent walls.

The present invention also provides a tridimensional closed system, wherein the tridimensional closed system has internal surfaces with all the characteristics of a mirror, and wherein the tridimensional closed system houses, while incubating, one or more containers with transparent walls containing the culture medium with differentiated cells or mycelia, and wherein the tridimensional closed system has a source of light with a determined wave length; and wherein the incubation is made under agitation, and wherein the tridimensional closed system has a mechanism to monitor temperature, and a mechanism to regulate temperature.

In one aspect of the present invention the source is a light emitting diode (LED) which emits blue light.

In one further aspect of the present invention, the tridimensional closed system comprises a tridimensional enclosure that is constituted by one or more than one compartment, wherein each compartment has at least one source of light, and wherein the source of light of each compartment is a LED, and wherein the LED of each compartment emits a light with a determined wave length.

In another aspect of the present invention, biomass produced after incubating the container/s with transparent walls containing the culture medium with differentiated mycelia or cells, inside the tridimensional closed system, is purified and dried.

Objectives and additional advantages of the present invention will become more evident in the description of the figures, the detailed description of the invention and the claims.

### BRIEF DESCRIPTION OF THE DRAWINGS

FIG. 1. is an illustrative view of the tridimensional closed system of the present invention.

FIG. 2. is another view of the tridimensional closed system of the present invention, in which one of the walls of the tridimensional enclosure is hypothetically open to illustrate that all the internal surfaces (shaded areas) of the enclosure have all the characteristics of mirrors.

FIG. 3 shows the tridimensional closed system of the present invention, wherein the tridimensional enclosure has more than one compartment, wherein each compartment has one source of light.

### DETAILED DESCRIPTION OF THE INVENTION

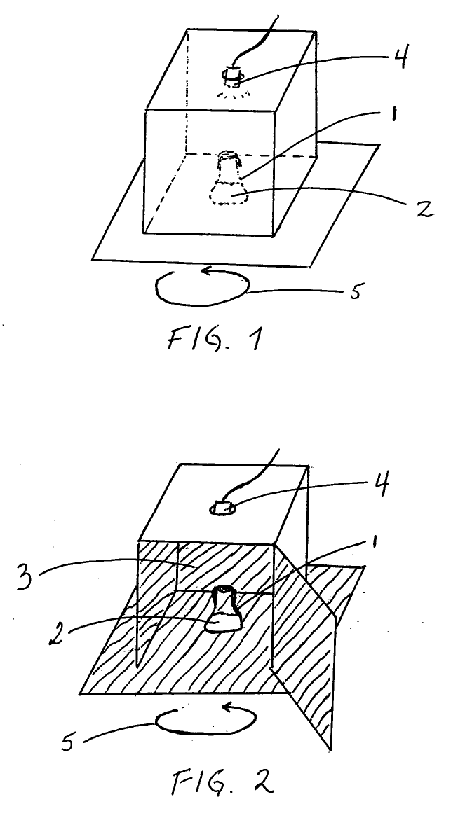

The present invention provides a method to produce biomass comprising:
A. Inoculating differentiated cells into a culture medium which contains at least a source of carbon;
B. Putting at least one container (1)(FIGS. 1 and 2) with transparent walls inside a tridimensional closed system (FIGS. 1 and 2), wherein the container (1) with transparent walls contains the culture medium (2) inoculated with differentiated cells, and wherein the tridimensional closed system has internal surfaces (3)(shaded areas of FIG. 2) with all the characteristics of a mirror, and wherein the closed system has a source of light (4) with a determined wave length;
C. Incubating until there is visible production of biomass; and,
D. Purifying and collecting said biomass;

wherein the container (1) with transparent walls containing the culture medium is agitated (5)(which indicates agitating motion), and wherein the closed system has a mechanism to monitor temperature and a mechanism to regulate temperature, and wherein the source of light (4) is at least one light emitting diode (LED) (4) which emits a light with a determined wave length, and wherein the wave length can be any wave length from the light spectrum.

The present invention also provides a tridimensional closed system (FIGS. 1 and 2) to incubate cell or mycelia cultures comprising:
a. A tridimensional enclosure with internal surfaces (3), wherein the internal surfaces have all the characteristics of a mirror, and wherein said tridimensional enclosure houses inside at least one container (1) with transparent walls, and wherein the container with transparent walls contains culture medium (2) with incubating cells; and,
b. A source of light (4) to the internal space of said tridimensional enclosure;

wherein the closed system has a mechanism to agitate (5) the container (2) with transparent walls, and wherein the tridimensional enclosure has a mechanism to monitor temperature and a mechanism to regulate temperature, and wherein the source of light (4) is at least one LED (4) which emits light with a determined wave length, and wherein the wave length can be any wave length from the light spectrum.

The definition of the tridimensional closed system of the present invention, in which all the internal walls of said system have all the characteristics of a mirror, includes all kind of enclosures with room or space, (e.g., incubators, bioreactors) to house any type of container for culturing cells, bacteria, mycelia etc.

The tridimensional closed system of the present invention may also have an attached mechanism to agitate the container or containers with transparent walls.

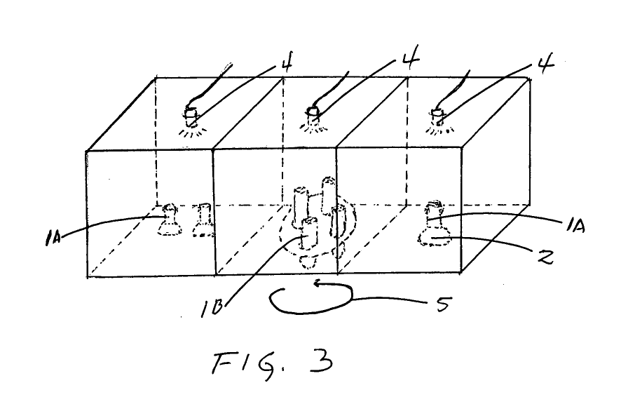

In one embodiment of the present invention the container with transparent walls is an Erlenmeyer flask (1A) (FIG. 3), while in another embodiment the container with transparent walls is a culture tube (1B)(FIG. 3). However, the container of the present invention can have any geometrical form, as long as, it has transparent walls, in order to allow the penetration of light inside of said container and therefore direct exposure of the culture medium being incubated to the light.

In another embodiment of the present invention, the tridimensional enclosure is constituted by a single compartment (FIGS. 1 and 2).

In another embodiment of the present invention, the tridimensional enclosure is constituted by more than one compartment (FIG. 3), wherein each compartment has at least one source of light (4), and wherein the source of light of each compartment is a LED (4), and wherein the LED of each compartment emits a light with a determined wave length, and wherein the wave length can be any wave length from the light spectrum.

The present invention further provides a method to produce biomass from fungi mycelium comprising:
1. A first inoculation of differentiated cells or mycelia into a solid culture medium which contains at least a source of carbon;
2. Incubating inside a first incubator the solid culture medium that has been inoculated with differentiated cells or mycelia for a determined number of days;
3. A second inoculation of the first inoculated solid culture medium, into a first liquid culture medium which contains at least NaNO₃, KH₂PO₄, MgSO₄, KCl, and a source of carbon;
4. Incubating by agitating inside a second incubator the first liquid culture medium that has been inoculated with first inoculated solid culture medium for a determined number of days;
5. A third inoculation of first liquid culture medium into a second liquid culture medium which contains at least NaNO₃, KH₂PO₄, MgSO₄, KCl, and a source of carbon;
6. Incubating by agitating inside a third incubator the second liquid culture medium that has been inoculated with the first liquid culture medium for a determined number of days; and,
7. Filtering and purifying, biomass produced within the second liquid culture medium that was previously inoculated with the first liquid culture medium;

wherein the solid culture medium contains at least agar, and a source of carbon in a concentration between 20 and 100 grams/Liter; and wherein, the pH of the liquid culture medium is adjusted between 4.5 and 7.0.

In an additional aspect of the present invention, regarding the method to produce biomass from fungi mycelium, the step of filtering and purifying of the biomass produced in the second liquid culture medium is performed by putting the biomass in a mesh that filters the excess liquid culture medium, and then, drying said biomass.

In a more preferred aspect of the present invention, regarding the method to produce biomass from fungi mycelium, the solid culture medium contains at least agar, and a source of carbon in a concentration of 50 grams/Liter; and wherein, the pH of the liquid culture medium is adjusted to 5.5±0.1.

In another preferred embodiment of the present invention, regarding the method to produce biomass from fungi mycelium, the source of carbon is a source selected from the group of barley flour, oats flour, rice flour, wheat flour, corn flour, and a mix of cereals. However, in a more preferred embodiment of the present invention, regarding the method to produce biomass from fungi mycelium, the source of carbon is barley flour or oats flour.

In one preferred aspect of the present invention, regarding the method to produce biomass from fungi mycelium, the fungi mycelium is from a fungus from a phyllum selected from the group consisting of basidiomycetes phyllum, and Ascomycetes phyllum. However, in a more preferred aspect of the present invention, regarding the method to produce biomass from fungi mycelium, the fungi mycelium is a fungus from the basidiomycetes phyllum.

More specifically, regarding the method to produce biomass from fungi mycelium, the fungi mycelium is preferably selected from a group consisting of Ganoderma lucidum, Ganoderma australe, Auricularia fuscosuccinea, Auricularia aurícula, Oudemansiella canarri, Agaricus blazei also known as Agaricus brasiliensis, Agaricus bisporus, Lentinula edodes, Grifola frondosa, Flammulina velutipes, Volvariella volvaceae, Amauroderma sp., Schysophyllum communis, Pleurotus ostreatus, Pleurotus pulmonarius, Pleurotus citrinopileatus, Pleurotus djamor, Pleurotus sp., Psilocybe cubensis, Coprinus sp., Polyporus sanguineus, Hericium erinaceus, Morchella sculenta, Gibberella fujikuroi, and Mycosphaerella fijiensis; However, the fungi mycelium is even more preferably from the fungus Ganoderma lucidum.

In another aspect of the present invention, regarding the method to produce biomass from fungi mycelium, both, the first liquid culture medium and the second liquid culture medium, contain at least NaNO₃ in a concentration of more than 59 mg/Liter, KH₂PO₄ in a concentration between 20 and 80 mg/Liter, MgSO₄ in a concentration between 10 and 60 mg/Liter; KCl in a concentration between 5 and 40 mg/Liter, and source of carbon in a concentration between 20 and 100 gramos/Liter, and wherein, the pH of the liquid culture medium is adjusted between 4.5 and 7.0.

In a more preferred aspect of the present invention, regarding the method to produce biomass from fungi mycelium, the first liquid culture medium and the second liquid culture medium, contain NaNO₃ in a concentration of 80 mg/Liter, KH₂PO₄ in a concentration of 30 mg/Liter, MgSO₄.H₂O in a concentration of 20 mg/Liter, KCl in a concentration of 10 mg/Liter, and a source of carbon in a concentration of 50 grams/Liter; and wherein, the pH of the liquid culture medium is adjusted to 5.5±0.1.

In another aspect of the present invention, regarding the method to produce biomass from fungi mycelium, incubating in the first incubator is for at least 15 days at a temperature between 15 and 35° C.; incubating in the second incubator is for at least 15 days at a temperature between 15 and 35° C.; and incubating in the third incubator is for at least 9 days at a temperature between 15 and 35° C.

In a preferred aspect of the present invention, regarding the method to produce biomass from fungi mycelium, incubating in the first incubator, in the second incubator and in the third incubator is at room temperature or at 25±1° C.

In another aspect of the present invention, regarding the method to produce biomass from fungi mycelium, the third incubator is a tridimensional closed system comprising:
i. A tridimensional enclosure with internal surfaces wherein all the internal surfaces have all the characteristics of a mirror, and wherein said tridimensional enclosure houses inside at least one container with transparent walls, and wherein the container with transparent walls contains the second liquid culture medium that has been inoculated with the first liquid culture medium, and;
ii. A source of light to the internal space of said tridimensional enclosure; and,
iii. A mechanism to agitate the container with transparent walls;

wherein the third incubator is a tridimensional closed system with a mechanism to monitor temperature and a mechanism to regulate temperature.

In one aspect of the present invention, regarding the method to produce biomass from fungi mycelium, the third incubator is a tridimensional closed system, wherein the tridimensional enclosure is constituted by at least one compartment, and wherein the tridimensional enclosure has a source of light to the inside of said compartment, and wherein the source of light is a LED that emits a determined wave length, and wherein the wave length can be any wave length from the light spectrum.

In a preferred aspect of the present invention, regarding the method to produce biomass from fungi mycelium, the wave length emitted by the LED is between 425-475 nm.

In a more preferred aspect of the present invention, regarding the method to produce biomass from fungi mycelium, the wave length emitted by the LED is between 425-475 nm for Ganoderma lucidum.

In addition, the present invention provides a culture medium for the production of biomass from the culture of fungi mycelium comprising at least NaNO₃, KH₂PO₄, MgSO₄, KCl, and a source of carbon.

In a preferred aspect of the present invention, regarding the culture medium for the production of biomass from fungi mycelium, said culture medium contains at least NaNO₃ in a concentration of more than 59 mg/Liter, KH₂PO₄ in a concentration between 20 and 80 mg/Liter, MgSO₄ in a concentration between 10 and 60 mg/Liter; KCl in a concentration between 5 and 40 mg/Liter, and source of carbon in a concentration between 20 and 100 gramos/Liter, and wherein, the pH of the liquid culture medium is adjusted between 4.5 and 7.0.

In a more preferred aspect of the present invention, regarding the culture medium for the production of biomass from fungi mycelium, said culture medium contains NaNO₃ in a concentration of 80 mg/Liter, KH₂PO₄ in a concentration of 30 mg/Liter, MgSO₄.H₂O in a concentration of 20 mg/Liter, KCl in a concentration of 10 mg/Liter, and a source of carbon in a concentration of 50 grams/Liter; and wherein, the pH of the liquid culture medium is adjusted to 5.5±0.1.

In one preferred embodiment of the present invention, regarding the culture medium for the production of biomass from fungi mycelium, the source of carbon is a source selected from the group of barley flour, oats flour, rice flour, wheat flour, corn flour, and a mix of cereals. However, in a more preferred embodiment of the present invention, regarding the culture medium for the production of biomass from fungi mycelium, the source of carbon is barley flour or oats flour.

In one preferred aspect of the present invention, regarding the culture medium for the production of biomass from fungi mycelium, the fungi mycelium is from a fungus from a phyllum selected from the group consisting of basidiomrycetes phyllum, and Ascomycetes phyllum. However, in a more preferred aspect of the present invention, regarding the culture medium for the production of biomass from fungi mycelium, the fungi mycelium is a fungus from the basidiomycetes phyllum.

More specifically, regarding the culture medium for the production of biomass from fungi mycelium, the fungi mycelium is preferably selected from a group consisting of Ganoderma lucidum, Ganoderma australe, Auricularia fuscosuccinea, Auricularia aurícula, Oudemansiella canarii, Agaricus blazei also known as Agaricus brasiliensis, Agaricus bisporus, Lentinula edodes, Grifola frondosa, Flammulina velutipes, Volvariella volvaceae, Amauroderma sp., Schysophyllum communis, Pleurotus ostreatus, Pleurotus pulmonarius, Pleurotus citrinopileatus, Pleurotus djamor, Pleurotus sp., Psilocybe cubensis, Coprinus sp., Polyporus sanguineus, Hericium erinaceus, Morchella sculenta, Gibberella fujikuroi, and Mycosphaerella fijiensis; However, the fungi mycelium is even more preferably from the fungus Ganoderma lucidum.

While the description presents the preferred embodiments of the present invention, additional changes can be made in the form and disposition of the parts without distancing from the basic ideas and principles comprised in the claims.

### EXAMPLE 1

Materials and Methods

Microorganism and Maintaining Medium

The strain of Ganoderma lucidum (donated by Dr. S. T. Chang to Applicants—Laboratory of Vegetal Biologia, Universidad de Antioquia) was kept on a médium made of dextrose potato agar in Petri plates, incubated at 26° C. for 10 days and stored a 4° C.

Inóculo

Petri plates with the strain of Ganoderma lucidum were activated at a temperature of 26° C. during 24 hours prior to inoculation. Then, discs of 1 cm of diameter of mycelia and agar were carried to Erlenmeyer's flasks of 250 ml with 62.5 ml culture medium, under aseptic conditions in a cabin of laminar horizontal flow (Laserquim®).

Culture Medium

The used culture medium corresponds to the protocol developed for the fungi culture by the Laboratory of Vegetal Biology. Such culture consists of a basic medium with salts: NaNO₃, 80 mg/l; KH₂PO₄, 30 mg/l; MgSO₄.7H₂O, 20 mg/l; KCl, 10 mg/l, supplemented with barley flour or oats flour 50 g/l.

Conditions of Culture Medium

The culture medium was adjusted to a pH of 5.5±0.1 (pH-meter HI-221, Hanna Instruments®), then, it was sterilized (Pressure Sterilizer® 1941x) at 15 psi, 121° C. for 15 minutes. Then, the culture medium was inoculated with 1 cm discs of mycelia and agar under constant agitation at 100 rpm during 9 days at 25±1° C., under different light wave lengths.

Study Design to Evaluate LEDs Effects

To maximize the illumination of the culture medium, a box of 55 cm³ was made with all its internal surfaces covered with flat mirrors. A central perforation was made on the superior wall of the box in order to install different LEDs. A thermocouple (UNI-T®) was installed inside the box to monitor the temperature. The box was put covering an orbital agitator which upper surface was also covered with a flat mirror, and over which nine Erlenmeyer's flasks of 250 ml were put on. The Erlenmeyer's flasks contain the culture of Ganodeema lucidum.

Analytic Methods

A completely random design was used with 9 experimental units per treatment for a total of 45 experimental units. The results were analyzed with the Statgraphics® 4.0. statistical package. The statistical analysis includes ANOVA y Post-ANOVA tests.

Results

The culture medium and the temperature remain constant, to evaluate the effect of different wave length emitting LEDs over the biomass production. The source of carbon for the described culture medium was barley flour. Table 1 illustrates a comparison of the used culture medium with other culture mediums in the literature.

TABLE 1
Table 1. Costs of our Laboratory culture medium and
published culture mediums for Ganodema lucidum.

| Costs of culture medium | | | | |
|---|---|---|---|---|
| ($ Colombian | (US | Yield | Time | |
| Pesos) | Dollars) | (g/l-DW) | (days) | Publication |
| 9,832.00 | 4.27 | 6.7 | 10 | Fang & Zhong, 2002a |
| 8,691.00 | 3.77 | 8.7 | 14 | Fang & Zhong, 2000b |
| 9,856.00 | 4.28 | 21.9 | 14 | Tang & Zhong, 2002a |
| 9,856.00 | 4.28 | 22.1 | 17 | Tang & Zhong2002 b |
| 9,856.00 | 4.28 | 15.6 | 14 | Tang & Zhong 2003 |
| 185.00 | 0.08 | 29.0125 | 9 | Lab. Vegetal Biology |
| | | with LED | | Lab. Plant |
| | | (B*) | | Biotechnology, 2006 |

*Blue light - 425–475 nm wave length.

For Ganoderma lucidum, the yield/day in the Vegetal Biology Laboratory was 3.225 g/Liter, compared with the best result, 1.564 g/Liter, published in the literature. In addition, with the protocol used by Applicants in the Vegetal Biology Laboratory, the efficiency cost-wise, in the production of the mycelia biomass, increased more than 69 times (445 Colombian pesos or 0.19 USDollars/1 gram (Tang & Zhong, 2002) compared with 6.37 pesos or 0.0027 USDollars/1 gram in the Vegetal Biology Laboratory and Plant Biotechnology Laboratory).

Moreover, the mycelia biomass produced by Applicants in the Vegetal Biology Laboratory—Plant Biotechnology Laboratory has a better or comparable exo-polysaccharides (EPS), intra-polysaccharides (IPS), and ganoderic acid (AG) composition (EPS: 2.705±0.15 g/L, IPS: 2, 33±0.182 g/L, AG: 369, 67±12,73 mg/L), when compared with the best results in the literature as shown in the review by Wagner et al. (see Wagner, R., Mitchell, D. A, Sassaki, G. L., Lopes de Almeida Amazonas A. M., Berovic, M., Food Technology and Biotechnology, 41(4): 371-382 (2003)), which analyzes the Ganoderma lucidum mycelia biomass composition for exo-polysaccharides, intra-polysaccharides, and ganoderic acid (EPS: 1.71 g/L, IPS: 2.49 g/L, AG: 582 mg/L).

### EXAMPLE 2

Culture Medium

The culture medium consisting of a basic medium with salts: NaNO₃, 80 mg/l; KH₂PO₄, 30 mg/l; MgSO₄.7H₂O, 20 mg/l; KCl, 10 mg/l, supplemented with barley flour or oats flour 50 g/l; is been used by Applicants to maintain the following fungi strains in the Laboratory of Vegetal Biology:

### 1. Ganoderma lucidum

### 2. Ganoderma australe

### 3 Auricularia fuscosuccinea

### 4. Auricularia aurícula

### 5. Oudemansiella canarii

### 6. Agaricus blazei also known as Agaricus brasiliensis

### 7. Agaricus bisporus

### 8. Lentinula edodes

### 9. Grifola frondosa

### 10. Flammulina velutipes

### 11. Volvariella volvaceae

### 12. Amauroderma sp.

### 13. Schysophyllum communis

### 14. Pleurotus ostreatus

### 15. Pleurotus pulmonarius

### 16. Pleurotus citrinopileatus

### 17. Pleurotus djamor

### 18. Pleurotus sp.

### 19. Psilocybe cubensis

### 20. Coprinus sp

### 21. Polyporus sanguineus

### 22. Hericium erinaceus

### 23. Morchella sculenta

### 24. Gibberella fujikuroi

### 25. Mycosphaerella fijiensis

### REFERENCES

1. Fang Q. H., Zhong J. J., Submerged fermentation of higher fungus Ganoderma lucidum for production of valuable bioactive metabolites-ganoderic acid and polysaccharide. Biochemical Engineering Journal, 10: 61-65 (2002).
2. Fang Q. H., Tang Y. J., Zhong J. J., Significance of inoculation density control in production of polysaccharide and ganoderic acid by submerged culture of Ganoderma lucidum. Process Biochemestry, 37: 1375-1379 (2002).
3. Tang, Y. J., Zhong, J. J., Role of oxygen supply in submerged fermentation of Ganoderma lucidum for production of Ganoderma polysaccharide and ganoderic acid. Enzyme and Microbial Technology, 32: 478-484 (2003).
4. Tang, Y. J., Zhong J. J., Exopolysaccharide biosynthesis and related enzyme activities of the medicinal fungus, Ganoderma lucidum, grow on lactose in a bioreactor. Biotechnology letters, 24: 1023-1026 (2002).
5. Tang, Y. J., Zhong, J. J., Fed-batch fermentation of Ganoderma lucidum for hyperproduction of polysaccharide and ganoderic acid. Enzyme and Microbial Technology, 31: 20-28 (2002).
6. Wagner, R.; Mitchell, D. A, Sassaki, G. L., Lopes de Almeida Amazonas A. M., Berovic, M., Current Techniques for the cultivation of Ganoderma lucidum for the production of biomass, ganoderic acid and polysaccharides. Food Technology and Biotechnology, 41(4): 371-382 (2003).

1. A method to produce biomass comprising:
A. Inoculating cells into a culture medium which contains at least a source of carbon;
B. Putting at least one container with transparent walls inside a tridimensional closed system, wherein the container with transparent walls contains the culture medium inoculated with cells, and wherein the tridimensional closed system has all internal surfaces with all the characteristics of a mirror, and wherein the closed system has a source of light with a determined wave length;
C. Incubating until there is visible production of biomass; and,
D. Purifying and collecting said biomass.

2. The method of claim 1, wherein the container with transparent walls containing the culture medium is agitated.

3. The method of claim 1, wherein the closed system has a mechanism to monitor temperature.

4. The method of claim 1, wherein the closed system has a mechanism to regulate the temperature.

5. The method of claim 1, wherein the closed system has a source of light, wherein the source of light is at least one light emitting diode (LED) which emits a light with a determined wave length.

6. A tridimensional closed system to incubate cell cultures comprising:
A. A tridimensional enclosure with internal surfaces, wherein all the internal surfaces have all the characteristics of a mirror, and wherein said tridimensional enclosure houses inside at least one container with transparent walls, and wherein the container with transparent walls contains culture medium with incubating cells; and,
B. A source of light to the internal space of said tridimensional enclosure.

7. The tridimensional closed system of claim 6, wherein the system has a mechanism to agitate the container with transparent walls.

8. The tridimensional closed system of claim 6, wherein the tridimensional enclosure has a mechanism to monitor temperature.

9. The tridimensional closed system of claim 6, wherein the tridimensional enclosure has a mechanism to regulate temperature.

10. The tridimensional closed system of claim 6, wherein the source of light is at least one LED which emits light with a determined wave length.

11. The tridimensional closed system of claim 6, wherein the tridimensional enclosure is constituted by a single compartment.

12. The tridimensional closed system of claim 6, wherein the tridimensional enclosure is constituted by more than one compartment, and wherein each compartment has at least one source of light.

13. The tridimensional closed system of claim 10, wherein the tridimensional enclosure is constituted by more than one compartment, and wherein each compartment has at least one source of light, and wherein the source of light is a LED, and wherein the LED of each compartment emits a light with a determined wave length.

14. A method to produce biomass from fungi mycelium comprising:
A. A first inoculation of fungus cells into a solid culture medium which contains at least a source of carbon;
B. Incubating inside a first incubator the solid culture medium that has been inoculated with fungus cells for a determined number of days;
C. A second inoculation of the first inoculated solid culture medium, into a first liquid culture medium which contains at least NaNO₃, KH₂PO₄, MgSO₄, KCl, and a source of carbon;
D. Incubating by agitating inside a second incubator the first liquid culture medium that has been inoculated with first inoculated solid culture medium for a determined number of days;
E. A third inoculation of first liquid culture medium into a second liquid culture medium which contains at least NaNO₃, KH₂PO₄, MgSO₄, KCl, and a source of carbon;
F. Incubating by agitating inside a third incubator the second liquid culture medium that has been inoculated with the first liquid culture medium for a determined number of days; and,
G. Filtering and purifying, biomass produced within the second liquid culture medium that was previously inoculated with the first liquid culture medium.

15. The method of claim 14, wherein the solid culture medium contains at least agar, and a source of carbon in a concentration between 20 and 100 grams/Liter; and wherein, the pH of the liquid culture medium is adjusted between 4.5 and 7.0.

16. The method of claim 15, wherein the source of carbon is one source selected from the group consisting of barley flour, oats flour, rice flour, wheat flour, corn flour, and a mix of cereals.

17. The method of claim 14, wherein the fungi mycelium is from a fungus from a phyllum selected from the group consisting of basidiomycetes phyllum, and Ascomycetes phyllum.

18. The method of claim 14, wherein the fungi mycelium is from a fungus selected from the group consisting of Ganoderma lucidum, Ganoderma australe, Auricularia fuscosuccinea, Auricularia aurícula, Oudemansiella canarii, Agaricus blazei also known as Agaricus brasiliensis, Agaricus bisporus, Lentinula edodes, Grifola frondosa, Flammulina velutipes, Volvariella volvaceae, Amauroderma sp., Schysophyllum communis, Pleurotus ostreatus, Pleurotus pulmonarius, Pleurotus citrinopileatus, Pleurotus djamor, Pleurotus sp., Psilocybe cubensis, Coprinus sp., Polyporus sanguineus, Hericium erinaceus, Morchella sculenta, Gibberella fujikuroi, and Mycosphaerella fijiensis.

19. The method of claim 14, wherein both, the first liquid culture medium and the second liquid culture medium, contain at least NaNO₃ in a concentration of more than 59 mg/Liter, KH₂PO₄ in a concentration between 20 and 80 mg/Liter, MgSO₄ in a concentration between 10 and 60 mg/Liter; KCl in a concentration between 5 and 40 mg/Liter, and source of carbon in a concentration between 20 and 100 gramos/Liter, and wherein, the pH of the liquid culture medium is adjusted between 4.5 and 7.0.

20. The method of claim 14, wherein incubating in the first incubator is for at least 15 days at a temperature between 15 and 35° C., and wherein incubating in the second incubator is for at least 15 days at a temperature between 15 and 35° C., and wherein incubating in the third incubator is for at least 9 days at a temperature between 15 and 35° C.

21. The method of claim 14, wherein the third incubator is a tridimensional closed system comprising:
A. A tridimensional enclosure with internal surfaces wherein the internal surfaces have all the characteristics of a mirror, and wherein said tridimensional enclosure houses inside at least one container with transparent walls, and wherein the container with transparent walls contains the second liquid culture medium that has been inoculated with the first liquid culture medium, and;
B. A source of light to the internal space of said tridimensional enclosure; and,
C. A mechanism to agitate the container with transparent walls.

22. The method of claim 14, wherein the third incubator is a tridimensional closed system with a mechanism to monitor temperature.

23. The method of claim 14, wherein the third incubator is a tridimensional closed system with a mechanism to regulate temperature.

24. The method of claim 21, wherein the third incubator is a tridimensional closed system, wherein the tridimensional enclosure is constituted by at least one compartment, and wherein the tridimensional enclosure has a source of light to the inside of said compartment, and wherein the source of light is a LED that emits a determined wave length.

25. The method of claim 24, wherein the wave length emitted by the LED is between 425-475 nm.

26. A culture medium for the production of biomass from fungi mycelium comprising at least NaNO₃, KH₂PO₄, MgSO₄, KCl, and a source of carbon.

27. The culture medium of claim 26, where the culture medium contains at least NaNO₃ in a concentration of more than 59 mg/Liter, KH₂PO₄ in a concentration between 20 and 80 mg/Liter, MgSO₄ in a concentration between 10 and 60 mg/Liter; KCl in a concentration between 5 and 40 mg/Liter, and source of carbon in a concentration between 20 and 100 gramos/Liter, and wherein, the pH of the liquid culture medium is adjusted between 4.5 and 7.0.

28. The culture medium of claim 26, wherein the source of carbon is one source selected from the group consisting of barley flour, oats flour, rice flour, wheat flour, corn flour, and a mix of cereals.

29. The culture medium of claim 26, wherein the fungi mycelium is from a fungus from a phyllum selected from the group consisting of basidiomycetes phyllum, and Ascomycetes phyllum.

30. The culture medium of claim 26, wherein the fungi mycelium is from a fungus selected from the group consisting of Ganoderma lucidum, Ganoderma australe, Auricularia fuscosuccinea, Auricularia aurícula, Oudemansiella canarii, Agaricus blazei also known as Agaricus brasiliensis, Agaricus bisporus, Lentinula edodes, Grifola frondosa, Flammulina velutipes, Volvariella volvaceae, Amauroderma sp., Schysophyllum communis, Pleurotus ostreatus, Pleurotus pulmonarius, Pleurotus citrinopileatus, Pleurotus djamor, Pleurotus sp., Psilocybe cubensis, Coprinus sp., Polyporus sanguineus, Hericium erinaceus, Morchella sculenta, Gibberella fujikuroi, and Mycosphaerella fijiensis.
